(19) United States
(12) Patent Application Publication
(10) Pub. No.: US 2020/0270670 A1
(43) Pub. Date: Aug. 27, 2020

(54) CULTURE MEDIUM FOR SELECTIVELY ISOLATING BACTERIA OF GENUS CLOSTRIDIUM

(71) Applicant: JNC CORPORATION, Tokyo (JP)

(72) Inventors: Hajime TERAMURA, Kanagawa (JP); Aya OGURA, Kanagawa (JP)

(73) Assignee: JNC CORPORATION, Tokyo (JP)

(21) Appl. No.: 16/646,580

(22) PCT Filed: Jun. 4, 2018
(86) PCT No.: PCT/JP2018/021319

§ 371 Date: Mar. 12, 2020

(30) Foreign Application Priority Data
Sep. 13, 2017 (JP) 2017-175897

(51) Int. Cl. C12Q 1/04; C12N 1/20

(52) U.S. Cl. CPC C12Q 1/045; G01N 2333/33; C12N 1/20

(57) ABSTRACT

To provide a culture medium in which plural kinds of bacteria of genus Clostridium can be selectively detected on the same culture medium and identified for every species. A culture medium for detecting bacteria of genus Clostridium contains (a) cycloserine, (b) polymyxin B, (c) an aminoglycoside-based antibiotic, (d) sodium taurocholate and (e) a phosphatase substrate capable of releasing a colored chromogen compound.

### TECHNICAL FIELD

The invention relates to a culture medium for selecting and isolating bacteria of genus Clostridium to detect the bacteria.

### BACKGROUND ART

Bacteria of genus Clostridium are Gram-positive obligate anaerobic spore-forming bacteria. Among the bacteria, in particular, Clostridium perfringens (hereinafter, also referred to as “Cp”) or Clostridium difficile (hereinafter, also referred to as “Cd”) is known as pathogenic bacteria. Moreover, Clostridium sporogenes (hereinafter, also referred to as “Cs”) is a toxin non-producer but used as an indicator of food contamination.

Cp is indigenous bacteria in large intestine of a human or an animal and widely distributed also in soil, sewage, a river, the sea and so forth, and a large amount of food such as meat, fish and shellfish and vegetables is contaminated by Cp. Further, Cp produces enterotoxin being a toxin when a spore is formed, and the spore is not completely killed by heat treatment and is detected also from a cooked food or a processed food. Therefore, Cp often causes food poisoning, and detection thereof is emphasized also from a viewpoint of food sanitation and food safety (Non-patent literature Nos. 1 and 2).

Cd is known as opportunistic-infection bacteria in nosocomial infection in a hospital, elderly recuperation facilities or the like, and causes pseudomembranous colitis when abnormal growth of intestinal Cd is caused by administration of an antibiotic in an inpatient. In recent years, large-scale infection by highly virulent Cd has been frequently caused in Europe and the Unites States, which is deemed as a problem (Non-patent literature Nos. 1 and 2).

As a selective isolation culture medium of Cp, Tryptose Sulfite Cycloserine agar (TSC agar) containing cycloserine, polymyxin B and kanamycin, or the like is known. If an analyte is applied to the TSC agar, and then anaerobically cultured, Cp causes reduction of sulfite to form black colonies, and most of other bacteria of genus Clostridium including Cd are inhibited (Non-patent literature Nos. 3 and 4).

Moreover, as a selective isolation culture medium of Cd, Cycloserine Cefoxitin Fructose Agar (CCFA agar) or the like is known. If an analyte is applied to the CCFA agar, and then anaerobically cultured, Cd forms colonies of rough type, but growth of most of intestinal bacteria and so forth is inhibited by cycloserine and cefoxitin

(Non-patent literature No. 5).

### CITATION LIST

### Non Patent Literature

NPL 1: Standard methods of analysis in food safety regulation: Analytical Methods for Microorganisms, 2015, Japan Food Hygiene Association, pp. 412 to 428, issued Mar. 31, 2015.

NPL 2: Guidebook of Easy and Rapid Microbial Test Methods, under the editorship of Shizunobu IGIMI et al., Technosystem Co., Ltd., pp. 197 to 198, issued Nov. 16, 2013.

NPL 3: Merck TSC Agar Catalog (12th edition of the Merck Microbiology Manual).http://www.merckmillipore.com/JP/ja/product/TSC-agar,MDA_CHEM-1119 72

NPL 4: Shin Saikinbaichigaku Koza (New Bacterial Culture Media) II (second edition), under the editorship of Riichi Sakazaki, Kindai Shuppan Co., Ltd., pp. 48 to 50, issued Jan. 20, 1990.

NPL 5: BD CCFA

Catalog.https://www.bdj.co.jp/micro/products/1f3pro00000s7gwy.html

### SUMMARY OF INVENTION

### Technical Problem

Neither a culture medium in which plural kinds of bacteria of genus Clostridium, for example, Cp and Cd can be selectively detected on the same culture medium has been provided so far, nor the culture medium in which both thereof can be identified on the same culture medium has been obviously provided, and therefore tests have been required to be conducted separately in the culture media under culture conditions suitable for each. However, irrespective of a variety of a food analyte and a clinical analyte, quick and reliable detection of presence of the bacteria of genus Clostridium such as Cp or Cd which are liable to be harmful a human in the analyte is important.

In view of such a situation, the invention is contemplated for providing a culture medium in which plural kinds of bacteria of genus Clostridium can be selectively detected on the same culture medium and both can be identified.

### Solution to Problem

The present inventors have diligently continued to conduct study in order to solve the problems as described above. As a result, the present inventors have found that a culture medium having a specific composition can cause growth of plural kinds of bacteria of genus Clostridium, and further appearance properties of colonies for every species are different on the culture medium, and have completed the invention.

More specifically, the invention includes the items described below.

Item 1. Use of a culture medium for detecting bacteria of genus Clostridium, wherein the culture medium contains (a) cycloserine, (b) polymyxin B, (c) an aminoglycoside-based antibiotic, (d) sodium taurocholate and (e) a phosphatase substrate capable of releasing a colored chromogen compound.

Item 2. The use of item 1, wherein the culture medium further contains (f) sugar or sugar alcohol containing one or more kinds selected from the group of mannitol, fructose and melezitose.

Item 3. The use of item 1 or 2, wherein the culture medium further contains (g) lecithin.

Item 4. The use of any one of items 1 to 3, wherein the bacteria of genus Clostridium are selected from the group of Clostridium perfringens, Clostridium difficile and Clostridium sporogenes.

Item 5. A culture medium for detecting bacteria of genus Clostridium, containing (a) cycloserine, (b) polymyxin B, (c) an aminoglycoside-based antibiotic, (d) sodium taurocholate and (e) a phosphatase substrate capable of releasing a colored chromogen compound.

Item 6. The culture medium according to item 5, further containing (f) sugar or sugar alcohol containing one or more kinds selected from the group of mannitol, fructose and melezitose.

Item 7. The culture medium according to item 5 or 6, further containing (g) lecithin.

Item 8. The culture medium according to any one of items 5 to 7, wherein the bacteria of genus Clostridium are selected from the group of Clostridium perfringens, Clostridium difficile and Clostridium sporogenes.

Item 9. A method for detecting bacteria of genus Clostridium, including a step of inoculating an analyte into the culture medium according to any one of items 5 to 8, a step of culturing microorganisms contained in the analyte, and a step of detecting colonies of the microorganisms.

### Advantageous Effects of Invention

If a culture medium according to the invention is used, plural kinds of bacteria of genus Clostridium can be selectively grown and clearly detected and identified as colonies having different appearance properties for every species. Accordingly, the plural kinds of bacteria of genus Clostridium, for example, Cp and Cd in an analyte in an environment in which the analyte is contaminated with various germs, a food analyte therein and a clinical analyte therein can easily be detected and identified on the same culture medium.

### BRIEF DESCRIPTION OF DRAWINGS

FIG. 1 shows a photograph of colonies of Cp on a culture medium according to the invention.

FIG. 2 shows a photograph of colonies of Cd on a culture medium according to the invention.

FIG. 3 shows photographs of colonies of Cd, Cp and Cs on a culture medium according to the invention.

### DESCRIPTION OF EMBODIMENTS

A culture medium according to the invention contains (a) cycloserine, (b) polymyxin B, (c) an aminoglycoside-based antibiotic, (d) sodium taurocholate and (e) a phosphatase substrate capable of releasing a colored chromogen compound.

(a)The cycloserine is a cell wall synthesis inhibitor and can suppress growth of most of bacteria excluding bacteria of genus Clostridium. Therefore, the bacteria of genus Clostridium can be isolated without receiving an influence of foreign bacteria.

A content of the cycloserine in the culture medium according to the invention is preferably 1 to 1,000 mg/L, and further preferably 150 to 300 mg/L as a concentration during use (during growth of microorganisms, the same shall apply hereinafter).

(b) The polymyxin B can suppress Gram-negative bacteria. In addition, growth of the bacteria of genus Clostridium is not influenced.

A content of the polymyxin B in the culture medium according to the invention is preferably 1 to 100 mg/L, and further preferably 5 to 50 mg/L as the concentration during use.

(c) The aminoglycoside-based antibiotic can suppress growth of most of the bacteria excluding the bacteria of genus Clostridium. Specific examples of the aminoglycoside-based antibiotic preferably include kanamycin, gentamicin, tobramycin and streptomycin.

A content of the aminoglycoside-based antibiotic in the culture medium according to the invention is preferably 1 to 100 mg/L, and further preferably 5 to 50 mg/L as the concentration during use.

(d) The sodium taurocholate (bile acid) is an ingredient for promoting growth of Cd and facilitating formation of characteristic and clear colonies of rough type.

A content of the sodium taurocholate in the culture medium according to the invention is preferably 0.1 to 5 g/L, and further preferably 0.5 to 2 g/L as the concentration during use.

(e) The phosphatase substrate capable of releasing the colored chromogen compound is used in order to form the bacteria of genus Clostridium as colored colonies depending on the chromogen compound. In association with growth of the bacteria of genus Clostridium on the culture medium, the substrate is hydrolyzed by phosphatase owned by the bacteria, the colored chromogen compound is released to color the colonies. Specific examples of the phosphatase substrate capable of releasing the colored chromogen compound include 5-bromo-3-indolylphosphoric acid, 5-bromo-4-chloro-3-indolylphosphoric acid and 5-bromo-6-chloro-3-indolylphosphoric acid. When such a substrate is used, the released chromogen compound is required to be colored by causing oxidation condensation of the compound by returning a state to an aerobic state after culture. In addition thereto, specific examples of the phosphatase substrate preferably include 1-{2-[4-(dimethylamino)benzoyl]phenyl}-1H-indol -3-yl phosphate, disodium salt (Aldol 515-phospahte, made by Biosynth AG). When the material is used, the oxidation condensation of the released chromogen compound is unnecessary, and therefore the colonies can be colored during culture under anaerobic conditions. A content of the phosphatase substrate in the culture medium according to the invention is preferably 0.01 to 0.5 g/L, and further preferably 0.05 to 0.15 g/L as the concentration during use.

The culture medium according to the invention preferably further contains (f) sugar or sugar alcohol. Such sugar or sugar alcohol preferably contains one or more kinds selected from the group of mannitol (mannite), fructose and melezitose, and mannitol is further preferred in the group. Cd can cause assimilation of such sugar or sugar alcohol, and therefore growth of Cd is promoted, and Cd can be further easily detected. Moreover, pH of the culture medium around the colonies of Cd is reduced by assimilation of sugar or sugar alcohol by Cd, and release of the colored chromogen compound by acid phosphatase owned by Cd is promoted. In addition, Cp and Cs do not ordinarily cause assimilation of mannitol, fructose and melezitose.

Even if the culture medium does not contain sugar or sugar alcohol, the culture medium according to the invention can cause growth of the bacteria of genus Clostridium including Cd, and the bacteria can be identified as the colonies having different appearance properties (Cp: red colonies, Cd: colorless colonies, Cs: peach color colonies), respectively. However, when sugar or sugar alcohol is incorporated thereinto, both cause growth of the bacteria as the colonies which are colored and different in color tones (Cp: red colonies, Cd: orange colonies, Cs: peach color colonies), and therefore the bacteria are further easily detected and identified, and such a case is preferred.

A content of the sugar or sugar alcohol in the culture medium according to the invention is preferably 1 to 30 g/L, and further preferably 1 to 10 g/L as the concentration during use.

The culture medium according to the invention preferably further contains (g) lecithin. Thus, the lecithin is decomposed by lecithinase owned by Cp, cloudiness (opaque zone) is caused around the colonies of Cp (so-called egg-yolk reaction), and visibility of the colonies and identification from Cd are enhanced. In addition, neither Cd nor Cs owns the lecithinase, and therefore such cloudiness is not exhibited. Here, the lecithin is preferably egg-yolk lecithin. Moreover, the lecithin is ordinarily added to the culture medium according to the invention in the form of yolk. A content of the lecithin in the culture medium according to the invention is preferably 1 to 20 g/L, and further preferably 5 to 10 g/L as the concentration during use. Moreover, a content of the yolk when the lecithin is incorporated thereinto in the form of yolk is preferably 1 to 10% by mass, and further preferably 1 to 3% by mass as the concentration during use.

The culture medium according to the invention is ordinarily solid (including a gel-form). Therefore, the culture medium according to the invention preferably contains a gelling agent. Here, the gelling agent means a substance that causes swelling and gelation by containing water, and plays a role of a matrix for shaping the culture medium.

The gelling agent is ordinarily a polymer compound and may be a substance to be generally used in a solid medium for culturing the microorganisms, such as a viscosity-improving polysaccharide and an absorbent polymer. Specific examples thereof include agar, guar gum, xanthan gum, locust bean gum, gellan gum, polyvinyl alcohol, alkyl cellulose such as methyl cellulose and ethyl cellulose, carboxyalkyl cellulose such as carboxymethyl cellulose and carboxyethyl cellulose, and hydroxyalkyl cellulose such as hydroxymethyl cellulose and hydroxyethyl cellulose, and a mixture in combination of one kind or two or more kinds can be used. Moreover, with regard to magnitude (average molecular weight, degree of polymerization or the like) of the polymer compound, the compound in the general range when the compound is used in the solid culture medium for culturing the microorganisms only needs to be used. For example, polyvinyl alcohol having a weight-average molecular weight in the range of preferably 5,000 to 200,000 and a degree of saponification in the range of preferably 75 to 99%, and further preferably 85 to 90% can be used.

A content of the gelling agent in the culture medium according to the invention should be adjusted to a general range when the gelling agent is used in the solid culture medium for culturing the microorganisms as the concentration during use. For example, when polyvinyl alcohol having a weight-average molecular weight in the range of 5,000 to 200,000 and a degree of saponification in the range of 75 to 99% is used, a content is preferably 140 to 300 g/L, and further preferably 160 to 260 g/L as the concentration during use. Moreover, for example, when agar having a weight-average molecular weight in the range of 10,000 to 1,000,000 is used, a content is preferably 5 to 30 g/L, and further preferably 10 to 20 g/L as the concentration during use. The culture medium can be easily handled and shaped by adjusting the content to such a level.

The culture medium according to the invention may arbitrarily contain, in addition to the ingredients described above, an antibacterial substance, a nutritional ingredient, inorganic salts, any other saccharide, a viscosity improver and a pH adjuster.

Specific examples of the antibacterial substance include polylysine, protamine sulfate, glycine and sorbic acid.

As the nutritional ingredient, peptone, an animal meat extract, a yeast extract or a fish meat extract is preferred, for example.

Specific examples of the inorganic salts include inorganic acid metal salt such as sodium chloride, potassium dihydrogen phosphate, disodium hydrogenphosphate, magnesium sulfate and sodium thiosulfate, and organic acid metal salt such as sodium pyruvate, iron ammonium citrate and sodium citrate.

Specific examples of any other saccharide include glucose, lactose, sucrose, xylose, cellobiose and maltose.

Specific examples of the viscosity improver include starch and a derivative thereof, hyaluronic acid, an acrylic acid derivative, polyether and collagen.

Specific examples of the pH adjuster include sodium carbonate and sodium hydrogencarbonate.

In addition, a selective substance other than the substance described above, for example, an antibiotic such as cefoxitin, or a surfactant such as sodium dodecyl sulfate (SDS), Tween 80 and bile salt such as sodium cholate prevent growth of the bacteria of genus Clostridium in several cases, and therefore is preferably not substantially incorporated into the culture medium according to the invention. In particular, the cefoxitin preferably is not substantially incorporated thereinto because Cp has cefoxitin sensitivity. Here, an expression “is not substantially incorporated thereinto” means that a content thereof is preferably 0.1 mg/L or less, further preferably 0.001 mg/L or less, and still further preferably 0 mg/L as the concentration during use.

Moreover, the culture medium according to the invention does not ordinarily simultaneously contain an iron ion and a sulfite ion. The reason is that the colonies turn black in the presence of the combination thereof, and therefore two kinds of colonies become difficult to be identified.

In the culture medium according to the invention, from a viewpoint of growth of the bacteria of genus Clostridium, pH during preparation of the culture medium is preferably 6.0 to 8.0, and further preferably 7.0 to 7.4.

A form of the culture medium according to the invention is not particularly limited, and the culture medium can be prepared into a sheet-form simple dry culture medium or the like, in addition to the form in which the culture medium is solidified in a petri dish or the like.

Specific examples of the sheet-shaped simple dry culture medium include a sheet-form culture medium having a configuration formed by laminating a layer containing a porous material and a layer containing a gelling agent, as described in WO 97/24432 A. In the above case, the layer containing the gelling agent should be taken as the culture medium according to the invention.

In the culture medium according to the invention, the plural kinds of bacteria of genus Clostridium are grown as the colonies having different appearance properties for every species and can be clearly detected and identified. Moreover, in the culture medium according to the invention, the plural kinds thereof can also be selectively isolated from other bacteria. Therefore, the culture medium can be preferably used in the method for detecting the bacteria of genus Clostridium according to the invention, and the culture medium is preferably suitable for a method for detecting Cp, Cd and Cs, and further preferably suitable for a method for detecting Cp and Cd.

The method according to the invention includes the step of inoculating the analyte into the culture medium according to the invention, the step of culturing the microorganisms contained in the analyte, and the step of detecting the colonies of the microorganisms. Here, in the culturing step, the bacteria are preferably anaerobically cultured at 33 to 45° C. for 24 to 48 hours.

Specific examples of the analyte to be applied to the culture medium according to the invention include perishable food such as meat, fish and shellfish, vegetables and fruits, processed food and beverage such as cheese, lactic fermenting beverage and a fermented food, and also a clinical analyte such as feces, drinking water, fresh water, sea water and a wiping analyte in a cooking place, a hospital or the like. Moreover, a culture fluid obtained by preculturing the analytes in an enrichment culture medium such as a thioglycollate medium and a cooked meat medium can also be used.

### EXAMPLES

Next, the invention will be described in greater detail by way of Examples, but the invention is not limited to the Examples.

(1) Preparation of Culture Medium

To 970 milliliters of purified water, 1 liter of amount of use of a composition ingredient shown in Table 1 was added, the resulting mixture was warmed and dissolved at 121° C. for 15 minutes and cooled to about 50° C. into a base medium, and the base medium was heated and sterilized. Cycloserine and kanamycin dissolved in purified water were filtered and sterilized and then added thereto, and well mixed. Further, Aldol(registered tradename)-515 phosphate (Biosynth AG) dissolved with dimethylsulfoxide was added thereto and well mixed (Table 2). Then, abacterially-collected yolk was added thereto to be 3% by mass in a final concentration, and well mixed. The resulting mixture was dispensed into a plastic petri dish (90 φmm) each by 15 mL and allowed to stand until the culture medium was solidified to prepare a culture medium according to the invention. The prepared culture medium was stored under an anaerobic state for two days or more in order to reduce a surface of the culture medium, and then used for a specimen test as described later.

TABLE 1

| | Final |
|---|---|
| <Base media> | concentration (g/L) |
| Proteose peptone No. 2 | 30 |
| Mannitol | 6 |
| Bacto yeast extract | 3 |
| Sodium taurocholate | 1 |
| Potassium dihydrogen phosphate | 1 |
| Disodium hydrogenphosphate | 5 |
| Sodium chloride | 2 |
| Magnesium sulfate | 0.1 |
| Agar | 15 |
| Polymyxin B | 0.01 |
| | (pH 7.2 ± 0.2) |

TABLE 2

| | Final |
|---|---|
| <Addition after sterilization> | concentration (g/L) |
| Cycloserine | 0.25 |
| Kanamycin | 0.01 |
| Aldol ™-515 phosphate | 0.1 |
| Yolk | 30 |
| | (pH 7.2 ± 0.2) |

(2) Provision of Strain

Among specimen strains, with regard to Cp, Cd and Cs, a material precultured in a sheep blood agar medium for 24 hours under anaerobic conditions was used as specimen bacteria. With regard to other strains, a material precultured in a sheep blood agar medium for 24 hours under aerobic conditions was used as specimen bacteria. Each specimen strain was streaked on the culture medium prepared in (1) by using a platinum loop, and after anaerobic culture at 35° C. for 48 hours, growth and appearance properties of colonies were confirmed.

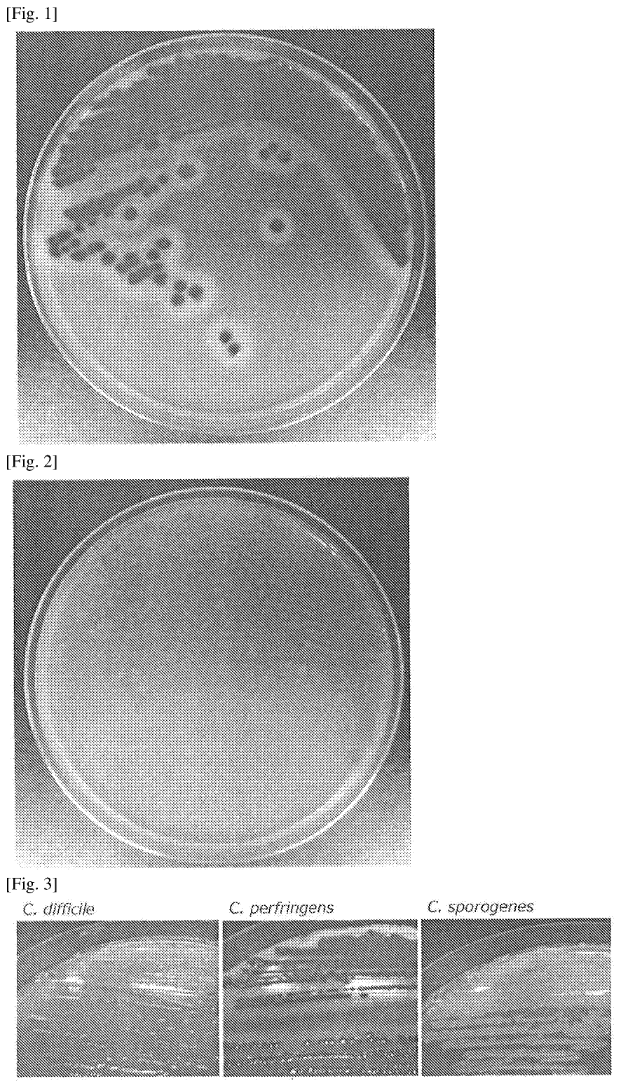

The results are shown in Table 3 and FIGS. 1 to 3.

TABLE 3

| Specimen bacteria | Colony growth state |
|---|---|
| Clostridium difficile JCM7571 | Colonies having an orange surface |
| | having a ground glass shape |
| Clostridium perfringens JCM3816 | Red colonies (cloudiness |
| | around the colonies) |
| Clostridium sporogenes NBRC13950 | Peach color metal-tone colonies |
| Escherichia coli NBRC102203 | Suppressed |
| Pseudomonas aeruginosa NBRC12689 | Suppressed |
| Bacillus subtilis NBRC3134 | Suppressed |
| Staphylococcus aureus NBRC 100910 | Suppressed |
| Candida albicans NBRC1594 | Suppressed |

Among the specimen strains, only Cp, Cd and Cs were able to grow on the culture medium according to the invention. Moreover, as shown in FIG. 1, Cp grew as clear red colonies having an opaque zone around the colonies, and as shown in FIG. 2, Cd was detected as orange colonies having no opaque zone around the colonies, and as shown in FIG. 3, Cs was detected as peach color metal-tone colonies having no opaque zone around the colonies, and the strains were able to be identified as the colonies having different appearance properties on the culture medium having the same composition. Moreover, also when an analyte in which plural kinds of bacteria of genus Clostridium coexist on the same culture medium was cultured, the bacteria were able to be identified as colonies having different appearance properties. Even with one kind of a phosphatase substrate capable of releasing a colored chromogen compound incorporated into the culture medium, the reason why a difference is caused in a color tone identifiable by the colonies of Cd, Cp and Cs is assumed to be caused by a difference in kinds of phosphatase owned by both strains.

Moreover, when Cp and Cd were provided as a specimen in a similar manner except that only mannitol was excluded from the composition in Table 1, Cp grew as red colonies, Cd grew as colorless colonies, and Cs grew as peach color colonies.

### INDUSTRIAL APPLICABILITY

The invention provides a culture medium in which plural kinds of bacteria of genus Clostridium can selectively grow and can be clearly detected and identified as colonies having different appearance properties. Thus, the bacteria of genus Clostridium in an analyte in an environment in which the analyte is contaminated with various germs, a food analyte therein and a clinical analyte therein can be easily detected and identified on the same culture medium, and therefore quick and simple detection of pathogenic bacteria harmful to a human body is achieved, and therefore such a culture medium is useful.

1. (orginal) Use of a culture medium for detecting bacteria of genus Clostridium, wherein the culture medium contains (a) cycloserine, (b) polymyxin B, (c) an aminoglycoside-based antibiotic, (d) sodium taurocholate and (e) a phosphatase substrate capable of releasing a colored chromogen compound.

2. (orginal) The use of claim 1, wherein the culture medium further contains (f) sugar or sugar alcohol containing one or more kinds selected from the group of mannitol, fructose and melezitose.

3. The use of claim 1 wherein the culture medium further contains (g) lecithin.

4. The use of claim 1, wherein the bacteria of genus Clostridium are selected from the group of Clostridium perfringens, Clostridium difficile and Clostridium sporogenes.

5. (orginal) A culture medium for detecting bacteria of genus Clostridium, containing (a) cycloserine, (b) polymyxin B, (c) an aminoglycoside-based antibiotic, (d) sodium taurocholate and (e) a phosphatase substrate capable of releasing a colored chromogen compound.

6. (orginal) The culture medium according to claim 5, further containing (f) sugar or sugar alcohol containing one or more kinds selected from the group of mannitol, fructose and melezitose.

7. The culture medium according to claim 5, further containing (g) lecithin.

8. The culture medium according to claim 5, wherein the bacteria of genus Clostridium are selected from the group of Clostridium perfringens, Clostridium difficile and Clostridium sporogenes.

9. A method for detecting bacteria of genus Clostridium, including a step of inoculating an analyte into the culture medium according to claim 5, a step of culturing microorganisms contained in the analyte, and a step of detecting colonies of the microorganisms.

10. The use of claim 2, wherein the culture medium further contains (g) lecithin.

11. The use of claim 2, wherein the bacteria of genus Clostridium are selected from the group of Clostridium perfringens, Clostridium difficile and Clostridium sporogenes.

12. The use of claim 3, wherein the bacteria of genus Clostridium are selected from the group of Clostridium perfringens, Clostridium difficile and Clostridium sporogenes.

13. The culture medium according to claim 6, further containing (g) lecithin.

14. The culture medium according to claim 6, wherein the bacteria of genus Clostridium are selected from the group of Clostridium perfringens, Clostridium difficile and Clostridium sporogenes.

15. The culture medium according to claim 7, wherein the bacteria of genus Clostridium are selected from the group of Clostridium perfringens, Clostridium difficile and Clostridium sporogenes.

16. A method for detecting bacteria of genus Clostridium, including a step of inoculating an analyte into the culture medium according to claim 6, a step of culturing microorganisms contained in the analyte, and a step of detecting colonies of the microorganisms.

17. A method for detecting bacteria of genus Clostridium, including a step of inoculating an analyte into the culture medium according to claim 7, a step of culturing microorganisms contained in the analyte, and a step of detecting colonies of the microorganisms.

18. A method for detecting bacteria of genus Clostridium, including a step of inoculating an analyte into the culture medium according to claim 8, a step of culturing microorganisms contained in the analyte, and a step of detecting colonies of the microorganisms.
